(19) United States
(12) Patent Application Publication
(10) Pub. No.: US 2005/0281763 A1
(43) Pub. Date: Dec. 22, 2005

(54) Personal care compositions comprising sparingly soluble solid oily components and solvents thereof

(76) Inventors: Yasuko Suginaka, Ashiya (JP); Hidekazu Tanaka, Toyonaka (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/153,284

(22) Filed: Jun. 15, 2005

Related U.S. Application Data
(60) Provisional application No. 60/580,524, filed on Jun. 17, 2004.

(51) Int. Cl. A61K 7/42

(52) U.S. Cl. 424/59

(57) ABSTRACT

Disclosed is a personal care composition comprising by weight: (a) from about 0.01% to about 10% of a sparingly soluble solid oily component; (b) from about 0.05% to about 50% of a solvent for the sparingly soluble solid oily component; (c) from about 0.01% to about 10% of an emulsifier; and (d) water; wherein the composition is an emulsion. The composition of the present invention provides reduced or eliminated crystals and precipitations of sparing soluble solid oily components.

### CROSS REFERENCE TO RELATED APPLICATION

This application claims the benefit of U.S. Provisional Application No. 60/580,524, filed on Jun. 17, 2004.

### FIELD OF INVENTION

The present invention relates to a personal care composition comprising: sparingly soluble solid oily components and solvents thereof. The composition of the present invention provides reduced or eliminated crystals and precipitations of sparing soluble solid oily components.

### BACKGROUND OF THE INVENTION

Many personal care products currently available to consumers are directed primarily to improving and/or maintaining the health and/or physical appearance of the skin.

For improving and/or maintaining the health and/or physical appearance of the skin, a variety of ingredients is used in the personal care products. For example, UV protecting agents are known to be advantageous for preventing excessive scaling and texture changes of the stratum corneum by exposure of ultraviolet light. Some UV protecting agents are also known to be solid oily components which are sparingly soluble in water and oil. Such sparingly soluble solid oily components are often contained in the compositions by solubilizing them in an oily phase in order to prevent uncomfortable gritty feel. Furthermore, when the sparingly soluble solid oily components are sparingly soluble solid oily UV protecting agents, it is preferred to dissolve them in a solvent for obtaining higher SPF.

It has been found that; even if such sparingly soluble solid oily components are contained in the composition by solubilizing them in an oily phase, such components form crystals or precipitations in the oily phase of the composition during the storage, for example, due to lower temperature during the storage. It has been found that; such sparingly soluble solid oily components form larger crystals or precipitations, especially when the composition is in the form of a water-in-oil emulsion. Such crystals and precipitations are not desirable in view of the product stability and/or the consumer acceptance, since they may cause the change of the product appearance and/or since they may cause uncomfortable gritty feel to the skin and/or hair when applied. Furthermore, when the sparingly soluble solid oily components are sparingly soluble solid oily UV protecting agents, such crystals and precipitations are not desirable in view of obtaining higher SPF.

Based on the foregoing, there exists a need for personal care compositions which provide reduced or eliminated crystals and precipitations of sparing soluble solid oily components.

None of the existing art provides all of the advantages and benefits of the present invention.

### SUMMARY OF THE INVENTION

The present invention is directed to a personal care composition comprising by weight:
(a) from about 0.01% to about 10% of a sparingly soluble solid oily component;
(b) from about 0.05% to about 50% of a solvent for the sparingly soluble solid oily component;
(c) from about 0.01% to about 10% of an emulsifier; and
(d) water;
wherein the composition is an emulsion.

The composition of the present invention provides reduced or eliminated crystals and precipitations of sparing soluble solid oily components. Thus, the composition of the present invention provides product stability and/or the consumer acceptance. When the sparingly soluble solid oily component is a sparingly soluble solid oily UV protecting agent, the composition can provide higher SPF.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

While the specification concludes with claims which particularly point out and distinctly claim the invention, it is believed the present invention will be better understood from the following description.

### COMPOSITION

The composition of the present invention is an emulsion. Such emulsions can be oil-in-water emulsions and water-in-oil emulsions. In the present invention, the compositions are preferably water-in-oil emulsions.

The composition of the present invention comprises a sparingly soluble solid oily component, solvent for the sparingly soluble solid oily component, an emulsifier, and water.

In the composition of the present invention, the weight ratio of the sparingly soluble solid oily component to the solvent is in the range of preferably from about 1:2 to about 1:10, more preferably from about 1:2 to about 1:5, still more preferably from about 1:3 to about 1:4.

Preferably, the compositions of the present invention are substantially free of non-polar oily components. Such non-polar oily components include silicone oils and hydrocarbons. In the present invention, “substantially free of non-polar oily components” means that the composition contain 3% or less, preferably 1% of non-polar oily components.

The composition of the present invention is suitable for topical use on human body, facial skin, and hair, and particularly suitable for facial skin. Typically, applications would be on the order of about once per day over such extended periods, while application rates can be up to about three times per day or more.

### SPARING SOLUBLE SOLID OILY COMPONENTS AND SOLVENTS THEREOF

The compositions of the present invention comprise a sparing soluble solid oily component, at a level of from about 0.01% to about 10%, preferably from about 0.1% to about 5.0%, more preferably from about 0.5% to about 3.0% by weight of the composition. The composition of the present invention comprises a solvent for the sparing soluble solid oily component, at a level of from about 0.05% to about 50%, preferably from about 0.5% to about 30%, more preferably from about 2.0% to about 20% by weight of the composition.

The sparing soluble solid oily components suitable for the use in the present invention are sold at 25° C., and have a low solubility in both oil and water. The sparing soluble solid oily components useful herein include, for example, sparing soluble solid oily UV protecting agents, ceramids, cholesterols, orizanols, and mixtures thereof. Among them, preferred are sparing soluble solid oily UV protecting agents.

Sparing Soluble Solid Oily UV Protecting Agent and Solvents Thereof

The UV protecting agents are those which generally prevent excessive scaling and texture changes of the stratum corneum by exposure of ultraviolet light. Sparing soluble solid oily UV protecting agents useful herein include, for example, butyl methoxy dibenzoylmethane (avobenzon), 2-hydroxy-4-methoxybenzo-phenone (oxybenzon), and octyltriazone. Among them, butyl methoxy dibenzoylmethane is highly preferred.

Such sparing soluble solid oily UV protecting agents are preferably used together with additional UV protecting agents. Such additional UV protecting agents useful herein include, for example, octyl methoxylcinnamate, octyl salicylate, octocrylene, homosalate, 2-phenylbenzimidazole-5-sulfonic acid, octyldimethyl-p-aminobenzoic acid, and 2-ethylhexyl N,N-dimethyl-p-aminobenzoate. Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978. Among the above additional UV protecting agents, liquid oily UV protecting agents such as octyl methoxylcinnamate, octyl salicylate, octocrylene, homosalate, and 2-ethylhexyl N,N-dimethyl-p-aminobenzoat, can be used as solvents of the sparingly soluble solid oily components.

The sparingly soluble solid oily UV protecting agents are dissolved in a solvent prior to the addition to the compositions. By dissolving the sparingly soluble solid oily UV protecting agents in a solvent, such UV protecting agents can provide higher SPF.

Such solvents are generally hydrophobic. Preferred are isononyl isononanoate; N-long-chain acyl neutral amino acid esters such as isopropyl lauroyl sarcosinate; phenethyl benzoate; butyloctyl salicylate; diethyl hexyl 2,6-naphthalate; tricaprylin; and mixtures thereof. In addition to these solvents, some of the additional UV protecting agents which are liquid oily UV protecting agents can be also used as solvents of the sparingly soluble solid oily components. Among a variety of solvents, preferred are isononyl isononanoate, isopropyl lauroyl sarcosinate, phenethyl benzoate, and mixtures thereof, and highly preferred is a mixture of isononyl isononanoate and isopropyl lauroyl sarcosinate, or a mixture of isononyl isononanoate and phenethyl benzoate. In the mixture, it is preferred that the weight ratio of isononyl isononanoate to isopropyl lauroyl sarcosinate or phenethyl benzoate is in the range of from about 5:1 to about 1:3, more preferably from about 3:1 to about 1:1, still more preferably 2:1.

N-long-chain acyl neutral amino acid esters are those having long-chain acyl group, natural amino acid moiety, and hydrocarbon group of the alcohol constituting the ester. The long-chain acyl group of N-long-chain acyl neutral amino acid esters is a straight-chain or branched-chain, saturated or unsaturated acyl group having from 6 to 22 carbon atoms. Examples thereof include those acyl groups which can be derived from capric acid, lauric acid, myristic acid, palmitic acid, stearic acid, behenic acid, linoleic acid, linolenic acid, oleic acid, isostearic acid, 2-ethylhexanoic acid, coconut oil fatty acid, tallow fatty acid, hardened tallow fatty acid, palm kernel oil fatty acid, and the like. Preferable examples of the acyl group include a caproyl group, a lauroyl group, a myristoyl group, a palmitoyl group, a stearoyl group, a behenoyl group, a coconut oil fatty acid acyl group, a hardened tallow fatty acid acyl group and the like. Further, examples of the neutral amino acids constituting the neutral amino acid moiety include neutral amino acids such as glycine, alanine, valine, leucine, isoleucine, serine, threonine, proline, beta -alanine, aminobutyric acid, sarcosine, N-methyl- beta -alanine and the like. Preferable are glycine, alanine, valine, leucine, isoleucine, beta -alanine, alpha -aminobutyric acid, gamma -aminobutyric acid, sarcosine and N-methyl- beta -alanine. More preferable are glycine, alanine, beta -alanine, alpha -aminobutyric acid, gamma -aminobutyric acid, sarcosine and N-methyl- beta -alanine. Further more preferable are sarcosine, alanine, glycine and N-methyl- beta -alanine. Especially preferable are N-alkyl neutral amino acids. Most preferable are sarcosine and N-methyl- beta -alanine. These amino acids may be optically active compounds or racemic compounds. The hydrocarbon group of the alcohol constituting the ester is a branched-chain or straight-chain alkyl or alkenyl group having from 1 to 10 carbon atoms. The alkyl group is preferable. Examples thereof include hydrocarbon groups which can be derived from ethanol, propanol, isopropanol, butanol, t-butanol, isobutanol, 3-methyl-1-butanol, 2-methyl-1-butanol, fusel oil, pentanol, hexanol, cyclohexanol, octanol, 2-ethylhexanol and decanol. A methyl ester having 1 carbon atom is undesirable because it means that methanol is used as a starting material. Further, a branched-chain or straight-chain alkyl group having from 2 to 8 carbon atoms is preferable from the standpoint of feeling upon use. Especially, a branched-chain or straight-chain alkyl group having from 2 to 5 carbon atoms is preferable because a dry or clean feeling is excellent and the effects of the present invention are exhibited more satisfactorily. Still further, a branched-chain alkyl group having from 3 to 5 carbon atoms is preferable from the aspect of the stability against hydrolysis in addition to the feeling upon use and the like. Examples thereof include an isopropyl group, a t-butyl group, an isobutyl group and the like. Among these, an isopropyl group is most preferable.

### EMULSIFIER

The compositions of the present invention comprise an emulsifier, at a level of from about 0.01% to about 10%, preferably from about 0.1% to about 5%, more preferably from about 0.5% to about 5% by weight of the composition.

Any emulsifiers can be used herein. Such emulsifiers useful herein include, for example, silicone copolyol esters, silicone copolyols, hydrophobically modified silicone copolyols, emulsifying crosslinked siloxane elastomer, polygrycerin esters, sucrose esters, and mixtures thereof. Among a variety of emulsifiers, preferred are hydrophobically modified silicone copolyols, emulsifying crosslinked siloxane elastomer, and mixtures thereof.

Silicone copolyol esters useful herein include, for example, those having from about 2 to about 10 dialkylsiloxane units preferably dimethylpolysiloxane units, having from about 2 to about 20 carbon atoms derived from a fatty acid, and having from about 5 to about 10 oxyalkylene groups. Such silicone copolyol esters include, for example, Dimethicone isostearate such as Dimethicone PEG-7 isostearate which is commercially available, for example, from Noveon with a tradename Ultrasil DW18, and Dimethicone olivate such as Dimethicone PEG-7 olivate which is commercially available, for example, from Noveon with a tradename Ultrasil DW-O.

Silicone copolyols, useful herein include, for example, those having from about 2 to about 10 dialkylsiloxane units preferably dimethylpolysiloxane units, and having from about 2 to about 10 oxyalkylene groups. Such silicone copolyol include, for example, Dimethicone copolyols such as PEG-10 Dimethicone which is commercially available, for example, from Shinetsu Chem. with a tradename KF-6017.

Hydrophobically Modified Silicone Copolyol

Hydrophobically modified silicone copolyols useful herein include, for example, those having from about 2 to about 10 dialkylsiloxane units preferably dimethylpolysiloxane units, having a hydrophobic substitution group having from about 2 to about 20 carbon atoms. Such hydrophobically modified silicone copolyols include, for example, Cetyl PEG/PPG-10/1 Dimethicone which is commercially available, for example, from Goldschmidt with a tradename Abil EM 90.

Emulsifying Crosslinked Siloxane Elastomer

Emulsifying crosslinked siloxane elastomers are also useful as the emulsifier of the present invention. The term “emulsifying,” as used herein, means crosslinked organopolysiloxane elastomer having at least one polyoxyalkylene unit. Emulsifying crosslinked organopolysiloxane elastomer can notably be chosen from the crosslinked polymers described in U.S. Pat. Nos. 5,412,004 (issued May 2, 1995); 5,837,793 (issued Nov. 17, 1998); and 5,811,487 (issued Sep. 22, 1998), all of which are herein incorporated by reference in their entirety.

Particularly useful emulsifying crosslinked siloxane elastomers are polyoxyalkylene modified crosslinked siloxane elastomers formed from divinyl compounds, particularly siloxane polymers with at least two free vinyl groups, reacting with Si—H linkages on a polysiloxane backbone. Preferably, the crosslinked siloxane elastomers are dimethyl polysiloxanes crosslinked by Si—H sites on a molecularly spherical MQ resin.

Highly preferred commercially available emulsifying crosslinked siloxane elastomers include, for example, PEG- 15 lauryl dimethicone crosspolymer which is supplied, for example, from Shinetsu Chemical with the tradenames KSG-320 and KSG-330. The emulsifying crosslinked siloxane elastomers are often supplied as a mixture with solvents.

### WATER

The compositions of the present invention comprise water. Deionized water is preferably used. Water from natural sources including mineral cations can also be used, depending on the desired characteristic of the product.

Generally, the composition of the present invention comprises from about 40% to about 99%, preferably from about 50% to about 90%, by weight of the composition, of water.

The pH of the present composition is preferably from about 4 to about 8, more preferably from about 5 to about 7. The suitable tacky skin treatment agents are particularly efficient in such pH range. Buffers and other pH adjusting agents can be included to achieve the desirable pH.

### WATER SOLUBLE HUMECTANT

The composition of the present invention may further contain from about 1% to about 20%, preferably from about 3% to about 10% of a water soluble humectant. Water soluble humectants useful herein include polyhydric alcohols such as glycerin, sorbitol, propylene glycol, butylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

Commercially available humectants highly useful herein include: butylene glycol with tradename 1,3-butylene glycol available from ASAHI DENKA Co. Ltd.; glycerin with tradenames CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; dipropylene glycol with the same tradename available from BASF; diglycerin with tradename DIGLYCEROL available from Solvay GmbH; polyethylene glycols with the tradename CARBOWAX series available from Union Carbide, and a mixture of glyceryl polymethacrylate, propylene glycol and PVM/MA copolymer with tradename Lubrajel Oil available from Guardian Lab.

### ESTER EMOLLIENT

The emollient oil useful herein are esters and those having a melting point of not more than about 25° C., and provide emollient benefit to the skin. Emollient oils of lower viscosity, low molecular weight, or branched structure, are highly preferable. It has been surprisingly found that, by the use of such oils, the tacky and greasy feel to the skin can be alleviated.

Emollient oils useful herein are esters, particularly esters having branched alkyl and alkenyl groups. Such esters include, for example, cetyl 2-ethylhexanoate, tridecyl isononanoate, isostearyl isostearate, isocetyl isostearate, isopropyl isostearate, isodecyl isononanoate, cetyl octanoate, diisopropyl myristate, isocetyl myristate, isotridecyl myristate, isopropyl myristate, myristyl myristate, isostearyl palmitate, isocetyl palmitate, isodecyl palmitate, isopropyl palmitate, isostearyl myristate, octyl palmitate, caprylic/capric acid triglyceride, glyceryl tri-2-ethylhexanoate, neopentyl glycol di(2-ethyl hexanoate), neopentyl glycol dicaprate, diisopropyl dimerate, glycerol trioctanate, glycerol triisopalmitate, isopropyl myristate, octyldodecyl lactate, and mixtures thereof. Triglycerides such as caprylic/capric triglyceride, PEG-6 caprylic/capric triglyceride, and PEG-8 caprylic/capric triglyceride may also be useful. Crude mixtures of such triglycerides by the CTFA name Meadowfoam seed oil are also useful. Commercially available oils include, for example, tridecyl isononanoate with tradename Crodamol TN available from Croda, and Hexalan available from Nisshin Seiyu, and Meadowfoam Seed Oil with tradename Cropure MDF available from Croda.

### TACKY SKIN TREATMENT AGENT

The composition of the present invention may contain from about 0.5% to about 10%, preferably from about 1% to about 5% of a tacky skin treatment agent. Skin treatment agents useful herein are those which help repair and replenish the natural moisture barrier function of the epidermis, thereby providing skin benefits such as texture improvement. It is generally known that, while such agents provide useful benefits to the skin when used chronically, they also tend to provide negative skin feel upon use when applied by itself.

Tacky skin treatment agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, urea, gelatin, soluble collagen, royal jelly, tocopheryl nicotinate, and vitamin D3 and analogues or derivatives, and mixtures thereof. Niacinamide is particularly preferred in that, when used in a pharmaceutically effective amount, is capable of reducing or alleviating the intensity of chronical spots. Niacinamide is suitably incorporated in the composition by first dissolving in water. Panthenol is also particularly preferred in that, when used in an amount of at least about 1%, it provides texture improvement benefits. Niacinamide and panthenol are commercially available, for example, by Roche.

### WHITENING AGENT

The composition of the present invention may further comprise from about 0.001% to about 10%, more preferably from about 0.1% to about 5% of a whitening agent. Whitening agents useful herein are those which are compatible with the aqueous form of the present composition. Water soluble whitening agents are preferred. The whitening agent useful herein refers to active ingredients that not only alter the appearance of the skin, but further improve hyperpigmentation as compared to pre-treatment.

Useful whitening agents useful herein include ascorbic acid compounds, azelaic acid, butyl hydroxy anisole, gallic acid and its derivatives, glycyrrhizinic acid, hydroquinoine, kojic acid, arbutin, mulberry extract, and mixtures thereof. Use of combinations of whitening agents is believed to be advantageous in that they may provide whitening benefit through different mechanisms.

Preferably, the ascorbic acid compound useful herein is an ascorbic acid salt or derivative thereof. Exemplary water soluble salt derivatives include, but are not limited to, L-ascorbic acid 2-glucoside, L-ascorbyl phosphate ester salts such as sodium L-ascorbyl phosphate, potassium L-ascorbyl phosphate, magnesium L-ascorbyl phosphate, calcium L-ascorbyl phosphate, aluminum L-ascorbyl phosphate. L-ascorbyl sulfate ester salts can also be used. Examples are sodium L-ascorbyl sulfate, potassium L-ascorbyl sulfate, magnesium L-ascorbyl sulfate, calcium L-ascorbyl sulfate and aluminum L-ascorbyl sulfate.

### ADDITIONAL COMPONENTS

The compositions herein may further contain other additional components, which may be selected by the artisan according to the desired characteristics of the final product and which are suitable for rendering the compositions more cosmetically or aesthetically acceptable or to provide them with additional usage benefits. The components useful herein are conveniently categorized by a certain benefit or their postulated mode of action, however, a given category is not limiting of its use. Further, it is understood the one component may provide multiple benefits.

(i) Anti-Oxidants and Radical Scavengers

Anti-oxidants and radical scavengers are especially useful for providing protection against UV radiation which can cause increased scaling or texture changes in the stratum corneum and against other environmental agents which can cause skin damage. Preferred anti-oxidants/radical scavengers include, for example, tocopherol sorbate and other esters of tocopherol, more preferably tocopherol sorbate. For example, the use of tocopherol sorbate in topical emulsions and applicable to the present invention is described in U.S. Pat. No. 4,847,071, Bissett et al, issued Jul. 11, 1989.

(ii) Anti-Inflammatory Agents

Anti-inflammatory agents enhance the skin appearance benefits, by for example, contribution of uniformity and acceptable skin tone and/or color. Preferably, the anti-inflammatory agent includes a steroidal anti-inflammatory agent and a non-steroidal anti-inflammatory agent. Preferred steroidal anti-inflammatory for use is hydrocortisone. So-called “natural” anti-inflammatory agents are also useful. For example, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may be used.

(iii) Antimicrobial Agent

As used, “antimicrobial agents” means a compound capable of destroying microbes, preventing the development of microbes or preventing the pathogenic action of microbes. Antimicrobal agents are useful, for example, in controlling acne. Preferred antimicrobial agents useful in the present invention are benzoyl peroxide, erythromycin, tetracycline, clindamycin, azelaic acid, sulfur resorcinol, phenoxyethanol, and Irgasan™ DP 300 (Ciba Geigy Corp., U.S.A.). A safe and effective amount of an antimicrobial agent may be added to emulsions of the present invention, preferably from about 0.001% to about 10%, more preferably from about 0.01% to about 5%, still more preferably from about 0.05% to about 2%.

(iv) Chelators

As used herein, “chelator” refers to a compound that reacts for removing a metal ion from a system by forming a complex so that the metal ion cannot readily participate in or catalyze chemical reactions. The inclusion of a chelator is especially useful for providing protection against UV radiation which can contribute to excessive scaling or skin texture changes and against other environmental agents which can cause skin damage. Exemplary chelators that are useful herein are disclosed in U.S. Pat. No. 5,487,884, Bissett et al, issued Jan. 30, 1996; PCT application 91/16035 and 91/16034, Bush et al, published Oct. 31, 1995. Preferred chelators are furildioxime and derivatives thereof.

(v) Sebum Absorbing Agent

The composition of the present invention may contain from about 0.1 % to about 10%, preferably from about 1% to about 5% of a sebum absorbing agent. Sebum absorbing agents useful herein include those which actually absorb the sebum discreted from the pores, and are compatible with the aqueous composition of the present invention. Components which are water soluble, water swellable, or have high emulsifying ability are not suitable herein, as they would no longer have sebum absorbing ability when formulated in the composition.

Preferable sebum absorbing agents herein include porous spherical cellulose powder, solid silicone elastomer powder, surface modified porous silica powder, porous nylon powder, porous acrylate copolymer, and mixtures thereof. The type and amount of sebum absorbing agents are selected according to the desired character of the product.

Commercially available porous spherical cellulose powders highly useful herein include the materials with tradename Celluflow series, such as Celluflow C025 available from Chisso Corp. Commercially available solid silicone elastomer powders highly useful herein include vinyl dimethicone/methicone silsesquioxane crosspolymer with tradenames KSP series available from ShinEtsu Chemical Co., Ltd., Tokyo Japan. Other commercially available sebum absorbing agents include porous acrylate copolymers with tradename Polytrap available from Dow Coming.

(vi) Sebum Suppressing Plant Extract

The composition of the present invention may further comprise from about 0.001% to about 5%, more preferably from about 0.05% to about 1% of a sebum suppressing plant extract. The plant extracts useful herein are those which have an astringent type of effect for reducing the size of pores, or inhibition effect of 5-α-reductase, and are compatible with the aqueous form of the present composition, and preferably do not alter the transparent or translucent appearance of the present composition. Water soluble plant extracts are preferred. Useful plant extracts herein include clove (choji) extract, coix (yokuinin) extract, witch hazel (hamamerisu) extract, and mixtures thereof. Such plant extracts are available from Iwase.

(vii) Other Components

In addition to the above described components, the composition of the present invention may further include preservatives and preservative enhancers such as water-soluble or solubilizable preservatives including Germall 115, methyl, ethyl, propyl and butyl esters of hydroxybenzoic acid, benzyl alcohol, imidazolidinyl urea, EDTA and its salts, Bronopol (2-bromo-2-nitropropane-1,3-diol) and phenoxypropanol; antifoaming agents; binders; biological additives; bulking agents; coloring agents; essential oils and solubilizers thereof; other natural extracts; compounds which stimulate collagen production; yeast fermented filtrates, and others.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below. All percentages and weights are based on the active level and, therefore, do not include carriers or by-products that may be included in commercially available materials.

Compositions

| Phase | | Ex. 1 | Ex. 2 | Ex. 3 | Ex. 4 | Ex. 5 |
|---|---|---|---|---|---|---|
| 01 | Emulsifier-1 *1 | 3.0 | 4.0 | 4.0 | — | 4.0 |
| 01 | Emulsifier-2 *2 | 1.0 | 0.5 | — | 1.0 | 1.0 |
| 01 | Emulsifier-3 *3 | — | — | — | 4.0 | — |
| 01 | Emulsifier-4 *4 | — | — | 1.0 | — | — |
| 02 | Butyl Methoxy | 2.0 | 2.0 | 2.0 | 2.0 | 3.0 |
| | Dibenzoylmethane | | | | | |
| 02 | Octyl Salicylate | 4.0 | 4.0 | 4.0 | 4.0 | 5.0 |
| 02 | Octocrylene | 1.3 | 1.3 | 1.3 | 1.3 | 2.0 |
| 02 | Isopropyl lauroyl | 2.0 | — | 2.0 | 3.0 | 4.0 |
| | sarcosinate | | | | | |
| 02 | Isononyl Isononanoate | 4.6 | 4.6 | 4.0 | 3.0 | 2.0 |
| 02 | Phenethyl Benzoate | — | 2.0 | — | — | — |
| 02 | Isopropyl Isostearate | — | — | 2.0 | — | — |
| 02 | Ethylparaben | 0.1 | 0.1 | 0.1 | 0.1 | 0.1 |
| 02 | Propylparaben | 0.1 | 0.1 | 0.1 | 0.1 | 0.1 |
| 03 | Perfume | 0.1 | 0.1 | 0.1 | 0.1 | 0.1 |
| 04 | Polymethyl- | — | 3.0 | — | — | — |
| | silesquioxane *5 | | | | | |
| 05 | Triethanol amine | 0.5 | 0.5 | 0.5 | 0.5 | 0.8 |
| 05 | Phenylbenzimidazol | 1.0 | 1.0 | 1.0 | 1.5 | — |
| | Sulfonic Acid | | | | | |
| 05 | Niacinamide | — | — | 0.1 | — | — |
| 05 | D-panthenol | — | — | 0.5 | — | — |
| 05 | Acetyl Glucosamine | — | — | 0.5 | — | — |
| 05 | Glycerin | 5.0 | 5.0 | 5.0 | 6.0 | 2.0 |
| 05 | 1,3-Butylene glycol | — | — | — | 4.0 | 5.0 |
| 05 | 1,2-Pentanediol | 3.0 | — | — | — | — |
| 05 | Di-sodium EDTA | 0.1 | 0.1 | 0.1 | 0.1 | 0.1 |
| 05 | Benzyl Alcohol | 0.4 | — | — | — | — |
| 05 | Sodium Citrate | 0.1 | 0.1 | 0.1 | 4.0 | 0.1 |
| 05 | Sodium Chloride | 0.5 | 0.5 | 0.5 | 0.5 | 0.5 |
| 05 | Zizyphus Jujuba | — | — | — | — | 0.1 |
| | Fruit Extract *6 | | | | | |
| 05 | Witch Hazel | — | — | — | — | 0.1 |
| | Extract *7 | | | | | |
| 05 | Clove Extract *8 | — | — | — | — | 0.1 |
| 05 | Magnesium Ascorbyl | — | — | — | 3.0 | — |
| | Phosphate | | | | | |

| | |
|---|---|
| De ionized Water | Balance to 100% |

Definitions of Components
*1 Emulsifier-1: KSG-320 available from Shinetsu Chemical which is a mixture of PEG-15 Lauryl Dimethicone Crosspolymer and Isododecane
*2 Emulsifier-2: Cetyl PEG/PPG-10/1 Dimethicone available from Goldschmidt with tradename Abil EM 90
*3 Emulsifier-3: KSG-330 available from Shinetsu Chemical which is a mixture of PEG-15 Lauryl Dimethicone Crosspolymer and Triethylhexanoin
*4 Emulsifier-4: PEG-10 Dimethicone available from Shinetsu Chem with tradename KF-6017
*5 Polymethylsilesquioxane: Tospearl 145A available from GE Toshiba Silicone
*6 Zizyphus Jujuba Fruit Extract: Available from Ichimaru Pharcos
*7 Witch Hazel Extract: Hamamerisu Liquid available from Iwase
*8 Clove Extract: Choji Extract BG available from Iwase

Method of Preparation

The skin care compositions of “Ex. 1” through “Ex. 5” as shown above can be prepared any conventional method well know in the art. For example, the compositions of the present invention can be prepared by following steps:
(a) Heat Phase 03 to about 80° C. and mix until sparingly soluble solid oily UV protecting agents are completely dissolved. Then cool the Phase 03 to about 25° C.
(b) Add Phase 01 to the mixture of (a).
(c) Add Phase 03 and Phase 04 to the mixture of (b).
(d) Finally, add Phase 05 to the mixture of (c) with mixing.

The embodiments disclosed and represented by the previous “Ex. 1” through “Ex. 5” are water-in-oil emulsions, and have many advantages. For example, the compositions of Examples 1 through 5 are particularly useful for providing UV protecting benefit to the skin. Furthermore, the compositions of Examples 1 through 5 can provide reduced or eliminated crystals and precipitations of sparing soluble solid oily components.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A personal care composition comprising by weight:
(a) from about 0.01% to about 10% of a sparingly soluble solid oily component;
(b) from about 0.05% to about 50% of a solvent for the sparingly soluble solid oily component;
(c) from about 0.01% to about 10% of an emulsifier; and
(d) water;
wherein the composition is an emulsion.

2. The personal care composition of claim 1 wherein the sparingly soluble solid oily component is a sparingly soluble solid oily UV protecting agent.

3. The personal care composition of claim 2 wherein the sparingly soluble solid oily UV protecting agent is selected from the group consisting of butyl methoxy dibenzoylmethane, 2-hydroxy-4-methoxybenzo-phenone, octyltriazone, and mixtures thereof.

4. The personal care composition of claim 3 wherein the sparingly soluble solid oily UV protecting agent is butyl methoxy dibenzoylmethane.

5. The personal care composition of claim 1 wherein the solvent is selected from the group consisting of isononyl isononanoate, isopropyl lauryl sarcosinate, phenethyl benzoate, and mixtures thereof.

6. The personal care composition of claim 5 wherein the solvent is a mixture of isononyl isononanoate and isopropyl lauryl sarcosinate or a mixture of isononyl isononanoate and phenethyl benzoate.

7. The personal care composition of claim 6 wherein the isononyl isononanoate and the isopropyl lauryl sarcosinate are contained at a level such that the weight ratio of the isononyl isononanoate to the isopropyl lauryl sarcosinate is from about 5:1 to about 1:3

8. The personal care composition of claim 7 wherein the isononyl isononanoate and the isopropyl lauryl sarcosinate are contained at a level such that the weight ratio of the isononyl isononanoate to the isopropyl lauryl sarcosinate is 2:1.

9. The personal care composition of claim 6 wherein the isononyl isononanoate and the phenethyl benzoate are contained at a level such that the weight ratio of the isononyl isononanoate to the phenethyl benzoate is from about 5:1 to about 1:3.

10. The personal care composition of claim 9 wherein the isononyl isononanoate and the phenethyl benzoate are contained at a level such that the weight ratio of the isononyl isononanoate to the phenethyl benzoate is 2:1.

11. The personal care composition of claim 1 wherein the sparingly soluble solid oily component and the solvent are contained at a level such that the weight ratio of the sparingly soluble solid oily component to the solvent is from about 1:2 to about 1:10.

12. The personal care composition of claim 1 wherein the emulsifier is selected from the group consisting of hydrophobically modified silicone copolyols, emulsifying crosslinked siloxane elastomers, and mixtures thereof.
